(19) United States
(12) Patent Application Publication
(10) Pub. No.: US 2020/0367784 A1
(43) Pub. Date: Nov. 26, 2020

(54) METHOD AND DEVICE FOR DIAGNOSING ARRHYTHMIA USING UWB RADAR

(71) Applicant: Industry-University Cooperation Foundation Hanyang University, Seoul (KR)

(72) Inventors: Sung Ho CHO, Seoul (KR); Yu Na LEE, Goyang-si, Gyeonggi-do (KR); Jeong Woo CHOI, Seoul (KR); Young Hyo LIM, Seongnam-si, Gyeonggi-do (KR)

(21) Appl. No.: 16/959,813

(22) PCT Filed: Dec. 31, 2018
(86) PCT No.: PCT/KR2018/016977

§ 371 Date: Jul. 2, 2020

(30) Foreign Application Priority Data
Jan. 2, 2018 (KR) 10-2018-0000249

(51) Int. Cl. A61B 5/05; A61B 5/00; G01S 13/02; G01S 7/41

(52) U.S. Cl. CPC A61B 5/05; G01S 7/415; G01S 13/0209; A61B 5/7235

(57) ABSTRACT

Disclosed are a method and device capable of diagnosing arrhythmia by a contactless method by using a UWB radar. The method for diagnosing arrhythmia using a UWB radar disclosed herein comprises: a step of extracting a radar signal corresponding to a heartbeat component, from radar signals received after being reflected from an examinee; a step of analyzing a frequency component of the radar signal corresponding the heartbeat component; and a step of determining if the examinee has arrhythmia, on the basis of the magnitude of the value of the ratio between the largest peak value and the second largest peak value which are included in the frequency component.

### BACKGROUND

### 1. Technical Field

The present invention relates to a method and device for diagnosing arrhythmia, more particularly to a method and device for diagnosing arrhythmia using a UWB radar in a non-contact manner.

### 2. Description of the Related Art

UWB refers to radio technology in which a frequency band of 500 MHz or higher is used or the value defined as the fractional bandwidth is 25% or higher. A fractional bandwidth refers to a bandwidth of a signal relative to a center frequency. That is, UWB refers to radio technology that uses a wideband frequency and provides various advantages such as high range resolution, permeability, strong resistance to narrowband noise, and coexistence with other frequency-sharing devices.

IR-UWB (impulse-radio ultra-wideband) radar (hereinafter referred to as UWB radar) technology utilizes a system that combines a radar with such UWB technology and refers to radar technology in which impulse signals of a very short duration having wideband properties are transmitted, reflected off objects and persons, and returned, where the returned signals are received and used to perceive the surrounding circumstances.

A UWB radar system may generate impulse signals having a time width of several nanoseconds to several picoseconds at a signal generator unit and emit the impulse signals through a transmission antenna in a wide-angle or narrow-band angle. The emitted signals may be reflected off various objects or persons in the environment, and the reflected signals may pass through the receiver antenna and an ADC to be converted into digital signals.

Due to the advantages of the UWB radar described above, research is under way in numerous fields for utilizing the UWB radar. Current research efforts for technology developments are conducted in various fields, including medical devices for measuring breathing rates, pulse rates, etc., portable radar devices for lifesaving efforts in disaster areas, people-counting devices for counting the number of people within a certain area, and the like. One such example is shown in Korean Patent Publication No. 10-2015-0085689.

In the related art, an electrocardiogram test, which involves analyzing heartbeat waveforms obtained by electrodes attached to a patient's skin for a certain amount of time, was used to diagnose a patient's arrhythmia. In such a case, a specialist such as a medical doctor would have to personally check the heartbeat waveform to determine whether or not there is arrhythmia, and the patient would have to visit the hospital to undergo the diagnosis, incurring burdens on the patient in terms of time and costs. Also, as electrodes are attached to the skin, this method may be difficult to use on patients having sensitive skin such as newborn infants or burn victims. Moreover, it may not be easy to test for arrhythmia in the course of everyday life.

As such, there is a need for a non-contact method of diagnosing arrhythmia that can conveniently diagnose arrhythmia in a patient during the course of everyday life.

### SUMMARY OF THE INVENTION

The present invention aims to provide a method and device for diagnosing arrhythmia in a patient by using a UWB radar.

An embodiment of the present invention, which is conceived to achieve the objective above, provides an arrhythmia diagnosis method using a UWB radar, where the arrhythmia diagnosis method includes: extracting a radar signal corresponding to a heartbeat component from radar signals reflected off and received from a patient; analyzing a frequency component of the radar signal corresponding to the heartbeat component; and determining whether or not the patient has arrhythmia according to a magnitude of a ratio value between a maximum peak value and a second largest peak value included in the frequency component.

Another embodiment of the present invention, conceived to achieve the objective above, provides an arrhythmia diagnosis method using a UWB radar, where the arrhythmia diagnosis method includes: extracting a radar signal corresponding to a heartbeat component from radar signals reflected off and received from a patient; generating frequency component information, which is associated with a frequency component for a received time of the received radar signals, for the radar signal corresponding to the heartbeat component; and determining whether or not the patient has arrhythmia according to a distribution pattern of peak values included in the frequency component.

Still another embodiment of the present invention, conceived to achieve the objective above, provides an arrhythmia diagnosis device using a UWB radar, where the arrhythmia diagnosis device includes: a signal input unit configured to receive input of radar signals reflected off and received from a patient; a heartbeat component extraction unit configured to extract a radar signal corresponding to a heartbeat component from the inputted radar signals; a frequency analysis unit configured to analyze a frequency component of the radar signal corresponding to the heartbeat component; an arrhythmia determination unit configured to determine whether or not the patient has arrhythmia according to a magnitude of a ratio value between a maximum peak value and a second largest peak value included in the frequency component; and an information output unit configured to output a result of the determination.

According to the present invention, a patient can be tested for arrhythmia by analyzing the frequency component of a radar signal corresponding to a heartbeat component.

Also, according to the present invention, arrhythmia can be diagnosed using a UWB radar in a non-contact manner, making it possible to test for arrhythmia simply during the course of everyday life and to monitor arrhythmia remotely. Moreover, the present invention can be used for diagnosing arrhythmia in patients with whom it is difficult to attach electrodes due to sensitive skin.

### BRIEF DESCRIPTION OF THE DRAWINGS

FIG. 1 is a diagram illustrating an arrhythmia diagnosis system using a UWB radar according to an embodiment of the present invention.

FIG. 2 is a diagram illustrating an arrhythmia diagnosis device according to an embodiment of the present invention.

FIG. 3 is a flowchart illustrating an arrhythmia diagnosis method according to an embodiment of the present invention.

FIG. 4 is a diagram illustrating an example of received UWB radar signals.

### DETAILED DESCRIPTION OF THE INVENTION

As the invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In describing the drawings, similar reference numerals are used for similar elements.

Certain embodiments of the present invention are described below in more detail with reference to the accompanying drawings.

A normal person and an arrhythmia patient show different spectrograms for a UWB radar signal. When measuring the spectrograms, a normal person show peak values that are concentrated at the frequencies of 50 Hz to 80 Hz, with small variations over time and an almost constant distribution. However, in the case of an arrhythmia patient, the peak values are very irregular and are distributed sporadically.

Also, for a normal person, the ratio value between the maximum peak value, i.e. the highest magnitude, and the second largest peak value, i.e. the second highest magnitude, appears at about 2, but for an arrhythmia patient, the ratio value between the maximum peak value and the second largest peak value is smaller at about 1. This is because, whereas a normal person has a regular heart rate so that the energy is concentrated on the frequency corresponding to the maximum peak value, a patient having arrhythmia has an irregular heart rate so that the energy is dispersed over frequencies corresponding to several peak values.

In this way, when considering radar signals reflected off an arrhythmia patient and radar signals reflected off a normal person in the frequency range, the signals exhibit different properties, and these properties can be used to diagnose arrhythmia in a patient. The following provides a more detailed description of a method for diagnosing arrhythmia using a UWB radar.

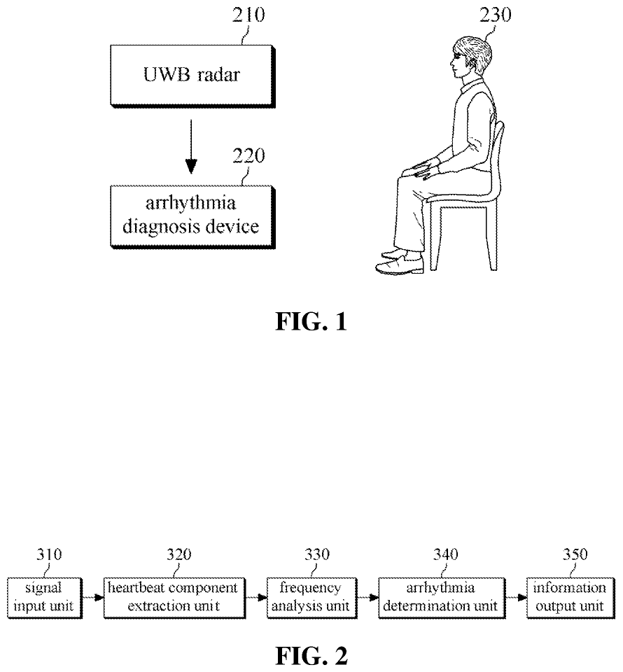

FIG. 1 is a diagram illustrating an arrhythmia diagnosis system using a UWB radar according to an embodiment of the present invention, and FIG. 2 is a diagram illustrating an arrhythmia diagnosis device according to an embodiment of the present invention.

Referring to FIG. 1, an arrhythmia diagnosis device 220 based on the present invention may use a radar signal reflected off and received from a patient 230 to provide a diagnosis as regards whether or not the patient 230 has arrhythmia. The UWB radar 210 may emit radar signals in the direction of the patient from a position separated by about 1-2 m from the patient 230 and may receive radar signals reflected off the patient 230. FIG. 2 illustrates an embodiment in which the UWB radar and the arrhythmia diagnosis device are used as separate devices, with the UWB radar providing the received radar signals to the arrhythmia diagnosis device, but depending on the embodiment, the UWB radar can be included in the arrhythmia diagnosis device.

Referring to FIG. 2, an arrhythmia diagnosis device 220 based on the present invention may include a signal input unit 310, a heartbeat component extraction unit 320, a frequency analysis unit 330, an arrhythmia determination unit 340, and an information output unit 350.

The signal input unit 310 may receive input of the radar signals reflected off and received from the patient 230, and the heartbeat component extraction unit 320 may extract the radar signals corresponding to the heartbeat component from the inputted radar signals.

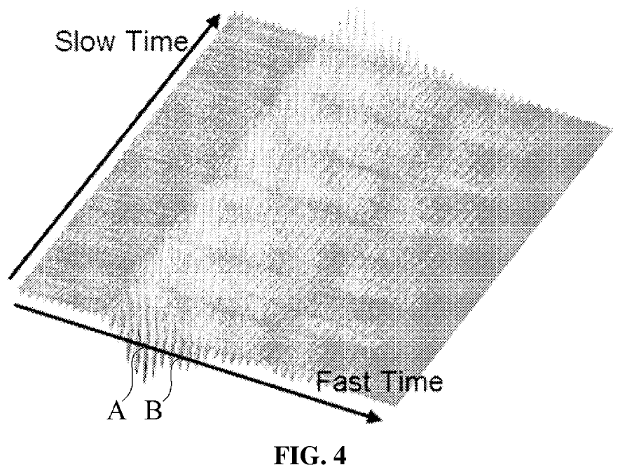

The frequency analysis unit 330 may analyze the frequency component of the radar signals corresponding to the heartbeat component to generate frequency component information. Since the radar signals are received continuously according to the passage of time and thus the radar signals corresponding to the heartbeat component are also signals that are continued over time, the frequency analysis unit 330 can generate the frequency component information according to the passage of time during which the radar signals corresponding to the heartbeat component are received. The frequency analysis unit 330 can analyze the frequencies for the radar signals corresponding to the heartbeat component in predetermined analysis segments, for instance in segments of 5 seconds, and can generate the frequency component information as defined for the time axis and frequency axis, such as in the spectrogram of FIG. 4.

The arrhythmia determination unit 340 may determine whether or not the patient 230 has arrhythmia according to the magnitude of the ratio value between the maximum peak value and the second largest peak value included in the frequency component. Alternatively, depending on the embodiment, the arrhythmia determination unit 340 can determine whether or not the patient 230 has arrhythmia according to the distribution pattern of peak values included in the frequency component.

The information output unit 350 can be a display device for outputting the arrhythmia determination result or can be a communication device such as a mobile terminal, etc., for transmitting the arrhythmia determination result.

The specific method for diagnosing arrhythmia is described below in further detail with reference to the drawings.

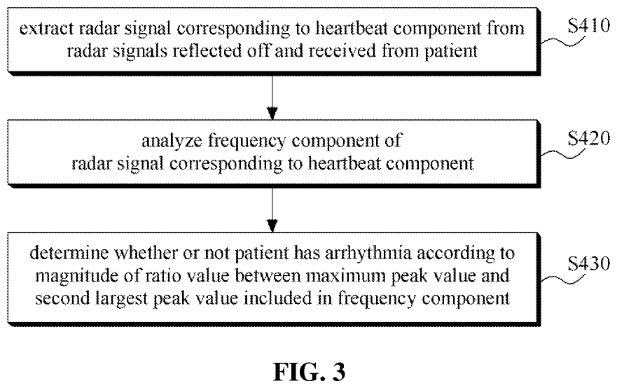

FIG. 3 is a flowchart illustrating an arrhythmia diagnosis method according to an embodiment of the present invention.

An arrhythmia diagnosis method based on the present invention can be performed by an arrhythmia diagnosis device that includes a processor, where the arrhythmia diagnosis device can be a device such as that of FIG. 1 or a computing device that includes a processor.

The radar signals transmitted over the UWB radar may be reflected off the patient and received at the UWB radar, and the arrhythmia diagnosis device based on the present invention may extract the radar signals corresponding to a heartbeat component from the radar signals reflected off and received from the patient (S410). The method of extracting the radar signals corresponding to the heartbeat component will be described in more detail later on with reference to FIG. 4.

Then, the arrhythmia diagnosis device based on the present invention may analyze the frequency component of the radar signals corresponding to the heartbeat component (S420).

Then, the arrhythmia diagnosis device based on the present invention may determine whether or not the patient has arrhythmia according to the magnitude of the ratio value between the maximum peak value and the second largest peak value included in the frequency component (S430). For example, the arrhythmia diagnosis device can determine that the patient is normal if the ratio value is greater than or equal to a threshold value and determine that the patient has arrhythmia if the ratio value is smaller than the threshold value. In another embodiment, the arrhythmia diagnosis device can determine that the patient has arrhythmia if the ratio value is smaller than a threshold value and close to 1, where the ratio value can be determined as being close to 1 if the ratio value is within a predetermined range from 1. Here, the threshold value can be set as 2 in one example, but this may vary depending on the embodiment.

In certain embodiments, an arrhythmia diagnosis device based on the present invention can additionally use a distribution pattern of the peak values included in the frequency component in step S420 in determining whether or not there is arrhythmia. As described above, the arrhythmia diagnosis device can generate frequency component information for the received time of the received radar signals in the manner of a spectrogram, and in one embodiment, the arrhythmia diagnosis device can determine that the patient has arrhythmia if a frequency for the peak values included in a predetermined time window lies beyond a predetermined frequency range. If the frequency for the peak values included in the time window is included in a predetermined frequency range, it can be determined that the patient is normal.

Here, the time window can correspond to the Y axis of the spectrogram and in one example can be 1 minute. In this case, the decision of whether or not a patient has arrhythmia can be determined from the frequency component for radar signals received during 1 minute. In step S430 also, it can be determined that the patient is normal if the ratio value between peak values included in the time window is greater than or equal to a threshold value and that the patient has arrhythmia if the ratio value is smaller than the threshold value.

Depending on the embodiment, an arrhythmia diagnosis device based on the present invention can use the method of determining arrhythmia using the ratio value and the method of determining arrhythmia using the distribution pattern of peak values either selectively or concurrently. When using the above arrhythmia determination methods concurrently, a final result can be determined when the result of determining arrhythmia using the ratio value and the result of determining arrhythmia using the distribution pattern of peak values agree with each other.

According to the present invention, arrhythmia can be diagnosed in a non-contact manner using a UWB radar, making it possible to diagnose arrhythmia in a simple manner during the course of everyday life as well as to monitor arrhythmia remotely. Also, the present invention can be used to diagnose arrhythmia for patients who have sensitive skin and thus have difficulties attaching electrodes.

In particular, instead of determining arrhythmia by analyzing only the frequency component of signals received at a specific time, the present invention can analyze the frequency component of signals received over a considerable duration of time corresponding to a time window, as in the spectrogram of FIG. 1, to determine the ratio values of peak values or the distribution patterns of peak values, so that the diagnosis of arrhythmia can be performed with greater accuracy.

FIG. 4 is a diagram illustrating an example of received UWB radar signals.

The radar signals reflected off and received from the patient can be expressed as a graph such as that shown in FIG. 4. In FIG. 4, the X axis (fast time) represents the distance between the UWB radar and the reflection point, and the Y axis (slow time) represents the received time. Also, the Y axis represents the amplitude of the signals. Each body part of the patient may have a different distance from the UWB, and each body part may vary in movement, so that the amplitudes of the radar signals reflected off different body parts of the patient may vary. For instance, a patient's chest area may show movement caused not only by respiration but also by heart movement, whereas other body parts may show movements caused only by respiration and not by heart movement.

The received radar signals may be of a form containing all of the signals reflected from all body parts of a patient, and for a diagnosing of arrhythmia, it may be necessary to extract the radar signals corresponding to the heartbeat component.

In order to extract the radar signals corresponding to the heartbeat component, an arrhythmia diagnosis device based on the present invention may calculate the difference between first signals for a first point of the patient and second signals for a second point of the patient and use the calculation results as the radar signals corresponding to the heartbeat component. Based on the fact that, from among the signals for each body part of the patient, signals affected both by respiratory motion and by heart rate motion show greater amplitudes compared to signals affected only by respiratory motion, the present invention may use signals having a relatively large amplitude as the first signals for a first point of the patient that include the heartbeat component and may use signals having a relatively small amplitude as the second signals for a second point of the patient that do not include the heartbeat component. For instance, the signals for point A on the X axis of FIG. 4 can be used as the first signals, while the signals for point B can be used as the second signals.

Since it cannot be ascertained which signals from among the received radar signals are the signals for the heart area of the patient that include the heartbeat component, an arrhythmia diagnosis device based on the present invention may use those signals having a relatively large amplitude as the signals reflected from the vicinity of the heart of the patient and may use those signals having a relatively small amplitude as the signals reflected from a point far away from the heart of the patient. Also, since signals having a very small amplitude would not be signals reflected off the patient, the first and second signals may be selected after determining that signals of a predetermined threshold or greater are signals reflected off arbitrary points of the patient.

In order to calculate the difference between the first signal and second signal, an arrhythmia diagnosis device based on the present invention may synchronize the first and second signals and project the second signal onto the first signal. Afterwards, the arrhythmia diagnosis device may calculate the difference between the first and second signals. Such a result may be very similar to the heartbeat waveform obtained by way of an ECG sensor.

The technical features described above can be implemented in the form of program instructions that may be performed using various computer means and can be recorded in a computer-readable medium. Such a computer-readable medium can include program instructions, data files, data structures, etc., alone or in combination. The program instructions recorded on the medium can be designed and configured specifically for the present invention or can be a type of medium known to and used by the skilled person in the field of computer software. Examples of a computer-readable medium may include magnetic media such as hard disks, floppy disks, magnetic tapes, etc., optical media such as CD-ROM's, DVD's, etc., magneto-optical media such as floptical disks, etc., and hardware devices such as ROM, RAM, flash memory, etc., configured specially for storing and executing program instructions. Examples of the program of instructions may include not only machine language codes produced by a compiler but also high-level language codes that can be executed by a computer through the use of an interpreter, etc. The hardware mentioned above can be made to operate as one or more software modules that perform the actions of the embodiments of the invention, and vice versa.

While the present invention is described above by way of limited embodiments and drawings that refer to particular details such as specific elements, etc., these are provided only to aid the general understanding of the present invention. The present invention is not to be limited by the embodiments above, and the person having ordinary skill in the field of art to which the present invention pertains would be able to derive numerous modifications and variations from the descriptions and drawings above. Therefore, it should be appreciated that the spirit of the present invention is not limited to the embodiments described above. Rather, the concepts set forth in the appended scope of claims as well as their equivalents and variations are encompassed within the spirit of the present invention.

What is claimed is:

1. An arrhythmia diagnosis method using a UWB radar, the arrhythmia diagnosis method comprising:
extracting a radar signal corresponding to a heartbeat component from radar signals reflected off and received from a patient;
analyzing a frequency component of the radar signal corresponding to the heartbeat component; and
determining whether or not the patient has arrhythmia according to a magnitude of a ratio value between a maximum peak value and a second largest peak value included in the frequency component.

2. The arrhythmia diagnosis method of claim 1, wherein the determining of whether or not the patient has arrhythmia comprises determining that the patient is normal if the ratio value is greater than or equal to a threshold value and determining that the patient has arrhythmia if the ratio value is smaller than the threshold value.

3. The arrhythmia diagnosis method of claim 2, wherein the threshold value is 2.

4. The arrhythmia diagnosis method of claim 1, wherein the determining of whether or not the patient has arrhythmia comprises further uses a distribution pattern of peak values included in the frequency component.

5. The arrhythmia diagnosis method of claim 4, wherein the determining of whether or not the patient has arrhythmia comprises determining that the patient has arrhythmia if a frequency for the peak values included in a predetermined time window lies beyond a predetermined frequency range.

6. The arrhythmia diagnosis method of claim 1, wherein the extracting of the radar signal corresponding to the heartbeat component comprises:
synchronizing a first signal for a first point of the patient with a second signal for a second point of the patient; and
calculating a difference between the first signal and the second signal,
and wherein the first signal has an amplitude greater than an amplitude of the second signal.

7. An arrhythmia diagnosis method using a UWB radar, the arrhythmia diagnosis method comprising:
extracting a radar signal corresponding to a heartbeat component from radar signals reflected off and received from a patient;
generating frequency component information for the radar signal corresponding to the heartbeat component, the frequency component information associated with a frequency component for a received time of the received radar signals; and
determining whether or not the patient has arrhythmia according to a distribution pattern of peak values included in the frequency component.

8. The arrhythmia diagnosis method of claim 7, wherein the determining of whether or not the patient has arrhythmia comprises determining that the patient has arrhythmia if a frequency for the peak values included in a predetermined time window lies beyond a predetermined frequency range.

9. An arrhythmia diagnosis device using a UWB radar, the arrhythmia diagnosis device comprising:
a signal input unit configured to receive input of radar signals reflected off and received from a patient;
a heartbeat component extraction unit configured to extract a radar signal corresponding to a heartbeat component from the inputted radar signals;
a frequency analysis unit configured to analyze a frequency component of the radar signal corresponding to the heartbeat component;
an arrhythmia determination unit configured to determine whether or not the patient has arrhythmia according to a magnitude of a ratio value between a maximum peak value and a second largest peak value included in the frequency component; and
an information output unit configured to output a result of the determination.

10. The arrhythmia diagnosis device of claim 9, wherein the arrhythmia determination unit determines that the patient is normal if the ratio value is greater than or equal to a threshold value within a predetermined time window and determines that the patient has arrhythmia if the ratio value is smaller than the threshold value.
